(19) United States
(12) Patent Application Publication
(10) Pub. No.: US 2024/0041307 A1
(43) Pub. Date: Feb. 8, 2024

(54) SCOPE WITH EXPANDABLE WORKING CHANNEL

(71) Applicant: GYRUS ACMI, INC. D/B/A OLYMPUS SURGICAL TECHNOLOGIES AMERICA, Westborough, MA (US)

(72) Inventors: Joey Magno, Dudley, MA (US); Jeffrey M. Schmitt, Bolton, MA (US)

(21) Appl. No.: 18/364,227

(22) Filed: Aug. 2, 2023

Related U.S. Application Data
(60) Provisional application No. 63/370,563, filed on Aug. 5, 2022.
(60) Provisional application No. 63/506,286, filed on Jun. 5, 2023.

(51) Int. Cl. A61B 1/018; A61B 1/307; A61B 1/015; A61B 1/05

(52) U.S. Cl. CPC A61B 1/018; A61B 1/307; A61B 1/015; A61B 1/05

(57) ABSTRACT

An endoscope may include an expandable and collapsible working channel, a flexible endoscope body, and an optical sensor located at a distal tip of the endoscope body. The optical sensor can provide a distal field of view from the distal tip. In the collapsed configuration, the working channel can be collapsed such that a channel wall is in a first position relative to the endoscope body and the working channel defines a first cross-sectional area. In the expanded configuration, the channel wall is displaced relative to the first position and the working channel defines a second cross-sectional area that is larger than the first cross-sectional area.

### CLAIM OF PRIORITY

This patent application claims the benefit of priority of Magno U.S. Provisional Patent Application Ser. No. 63/370,563, entitled “SCOPE WITH PASSIVE EXPANDABLE WORKING CHANNEL,” filed on Aug. 5, 2022 (Attorney Docket No. 5409.802PRV); and of Magno et al. U.S. Provisional Patent Application Ser. No. 63/506,286, entitled “SCOPE WITH PASSIVE OR ACTIVE EXPANDABLE WORKING CHANNEL,” filed on Jun. 5, 2023 (Attorney Docket No. 5409.829PRV) the benefit of priority of each of which is claimed hereby, and each of which are incorporated by reference herein in its entirety.

### INCORPORATION BY REFERENCE

This patent application incorporates by reference Magno et al., PCT Application Serial Number PCT/US2023/71489, entitled “SCOPE WITH EXPANDABLE WORKING CHANNEL,” filed on Aug. 2, 2023 (Attorney Docket No. 5409.802WO1) which is incorporated by reference herein in its entirety.

### TECHNICAL FIELD

This document pertains generally, but not by way of limitation, to a scope, for instance a cystoscope.

### BACKGROUND

Cystoscopy is a form of endoscopy that can be used for examining a region of interest of a patient, such as for instance the urethra or bladder. For example, cystoscopy can be used to diagnose suspected bladder cancer, an enlarged prostrate, or the like. A patient can undergo one or more of a PET scan, CT scan, urine cytology, MRI, or ultrasound, which can be useful to identify one or more biomarkers (e.g., a cyst, polyp, growth, tumor, tissue, biopsy site, or the like). Alternatively (or additionally), the patient can undergo a cystoscopy to identify the one or more biomarkers. A cystoscopy is more invasive than a PET scan, CT scan, urine cytology, MRI, or ultrasound imaging. For instance, during cystoscopy, a cystoscope can be inserted into a urethra of a patient. In contrast, an MRI is non-invasive. Patients can be apprehensive about undergoing the more invasive cystoscopy to identify biomarkers.

### SUMMARY

The present inventors have recognized, among other things, that a problem to be solved can include reducing or minimizing patient discomfort for a patient undergoing an endoscopy, for instance a cystoscopy. During a cystoscopy procedure, an endoscope that is sized with an outer diameter (or similar lateral outer dimension) that is similar to an inner diameter of a patient's urethra can be inserted into the urethra. However, the similarity in size between the endoscope and the urethra of the patient can cause discomfort for the patient undergoing the cystoscopy.

To help address this problem, an endoscope can be provided with an expandable and collapsible working channel. The expandable and collapsible working channel can help reduce or minimize the size (e.g., cross-sectional area, volume, or the like) of the n. For example, the endoscope can have a first lateral outer dimension size when the working channel is in a laterally collapsed configuration. The endoscope can have a second (larger) lateral outer dimension size when the working channel is in an expanded configuration. The working channel can be expanded, for instance to accommodate an instrument within the working channel.

For example, the endoscope can be provided in the laterally collapsed configuration. A user (e.g., technician, nurse, physician, surgeon, or the like) can insert the elongated endoscope into the urethra of the patient in the laterally collapsed configuration. This can help enhance patient comfort, such as during insertion, because the lateral size of the endoscope is smaller in the collapsed configuration than in the expanded configuration. For instance, the lateral size of the endoscope in the collapsed configuration can be dissimilar to (e.g., smaller than) the lateral size of the urethra of the patient. In the expanded configuration, the lateral size of the endoscope can be similar to that of the urethra. A patient may express discomfort with an endoscope having a similar lateral size as that of the urethra engaging with walls of the urethra of the patient, such as during insertion of the endoscope. Thus, inserting the endoscope in the smaller collapsed configuration can enhance patient comfort because the lateral size of the endoscope is smaller in the collapsed configuration, relative to the size of the urethra. Accordingly, the endoscope can reduce frictional engagement with walls of the urethra during insertion of the endoscope into the urethra of the patient. Translation of the endoscope in the collapsed configuration within the urethra can help reduce or minimize patient discomfort because frictional engagement of the endoscope with walls of the urethra can be minimized. Thus, the endoscope having an expandable and collapsible working channel can help reduce or minimize discomfort for a patient undergoing a cystoscopy, endoscopy, or the like.

Another problem to be solved can include enhancing accuracy of identifying biomarkers. For example, performing a cystoscopy to examine a patient can be more accurate in identifying biomarkers than non-invasive examination of the patient. A patient can be apprehensive about undergoing a cystoscopy due to the anticipated discomfort associated with the cystoscopy.

Using an endoscope having an expandable and collapsible working channel can help solve this problem. An endoscope having an expandable and collapsible working channel can help reduce or minimize patient discomfort during cystoscopy. This can help reduce apprehension of a patient to undergo a cystoscopy. Thus, the endoscope having an expandable and collapsible working channel can help enhance accuracy of biomarker identification because patient apprehension of the cystoscopy and discomfort during the cystoscopy can be reduced or minimized. This can make it easier for the patient to elect cystoscopy to identify the biomarkers. Cystoscopy can help avoid more invasive procedures to identify biomarkers, for instance a surgical procedure to identify the biomarkers. Enhancing patient adoption of cystoscopy can help reduce or minimize the need for more invasive procedures to identify biomarkers.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 shows a perspective view of an example of an endoscope.

FIG. 2 shows an end view of an example of a first end of another example of an endoscope.

FIG. 3 shows another end view of the first end of the endoscope of FIG. 2.

FIG. 4 shows a schematic diagram of the endoscope of FIG. 2.

FIG. 5 shows another schematic diagram of the endoscope of FIG. 2.

FIG. 6 shows a side view of the endoscope of FIG. 2.

FIG. 7 shows still another end view of the first end of the endoscope of FIG. 2.

FIG. 8 shows an end view of yet another example of the endoscope.

FIG. 9 shows another end view of the first end of the endoscope of FIG. 8.

FIG. 10 shows a side view of the endoscope of FIG. 8.

FIG. 11 shows an end view of still yet another example of the endoscope.

FIG. 12 shows first and second balloons in an inflated configuration.

FIG. 13 shows yet another example of the first end of the endoscope.

FIG. 14 shows an example of a method for using an endoscope.

### DETAILED DESCRIPTION

This document describes, among other things, an endoscope having an expandable and collapsible working channel, such as to provide a smaller lateral profile, which can help enhance patient comfort, such as during insertion of the endoscope. The cystoscopy can be used to help plan a medical procedure, for instance a transurethral resection of bladder tumor (“TURBT”). For example, the cystoscopy can be used to help identify one or more biomarkers for the TURBT. This can improve the cystoscopy procedure as an option, such as relative to other less invasive but also less accurate ways of identifying one or more biomarkers (e.g., a cyst, polyp, growth, tumor, tissue, biopsy site, or the like), such as using a PET scan, CT scan, urine cytology, MM, or ultrasound imaging. The findings of the non-invasive identification of one or more biomarkers can complement findings from the cystoscopy.

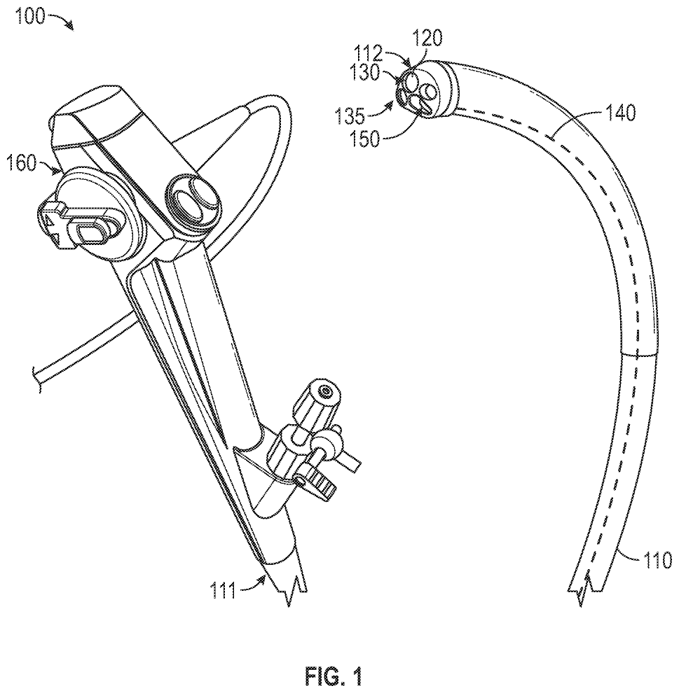

FIG. 1 shows a perspective view of an example of an endoscope 100. The endoscope 100 can be a cystoscope, a ureteroscope, or the like. The endoscope 100 can include an elongated endoscope body 110. For instance, the endoscope body 110 can extend between a proximal end 111 and a distal end 112. The endoscope body 110 can be flexible. The endoscope body 110 can be rigid. The endoscope can include an optical sensor 120. For instance, the endoscope 100 can include the optical sensor 120 located at a distal tip 130 of the endoscope body 110. The distal tip 130 can include a first end 135 of the endoscope 100. The optical sensor 120 can provide a distal field of view from the distal tip 130.

The endoscope 100 can include a working channel 140 (shown in dashed lines in FIG. 1). The working channel 140 can be expandable and collapsible. The working channel 140 can receive an instrument. The working channel 140 can extend longitudinally along the endoscope body 110. For instance, the working channel 140 can extend to a channel opening 150 located at the distal tip 130 of the endoscope body 110.

An instrument can be inserted into the working channel 140 of the endoscope 100. For instance, the endoscope 100 can include a proximal “control section” 160. The control section 160 can include one or more controls to operate the endoscope 100. A biopsy port 170 of the control section 160 can communicate with the working channel 140 of the endoscope 100. The biopsy port 170 may facilitate reception of an instrument within the working channel 140. For instance, an instrument inserted into the biopsy port 170 can travel through the working channel 140 to the distal tip 130. The instrument can extend (e.g., project, discharge, protrude, or the like) from the channel opening 150 at the distal tip 130 of the endoscope 100.

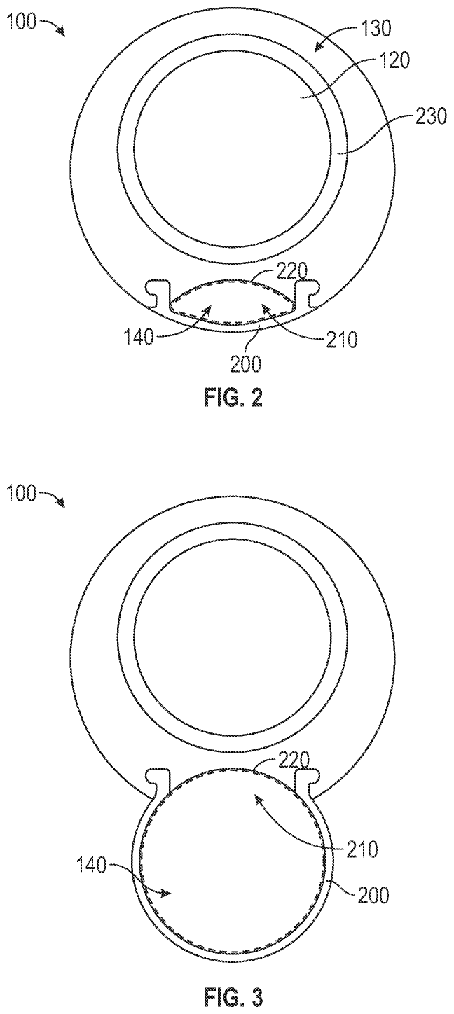

FIG. 2 shows an end view of the endoscope 110. The working channel 140 can have a collapsed configuration and an expanded configuration. In the collapsed configuration (shown in FIG. 2), the working channel 140 can be collapsed such that a channel wall 200 can be in a first position relative to an instrument socket 210 of the endoscope body 110. For instance, FIG. 2 shows the channel wall 200 in the first position relative to a socket wall 220 of the instrument socket 210. The channel wall 200 can have the first position in correspondence with an instrument outside (e.g., not received by, or the like) the working channel 140. In another example, the channel wall 200 can have the first position in correspondence with the instrument disengaged from the channel wall 200. In another example, FIG. 2 shows the channel wall 200 can be in a relaxed state. For instance, the channel wall 200 is relaxed with an instrument outside the working channel 140. The working channel 140 can have a first cross-sectional area (shown with dashed lines in FIG. 2) in the collapsed configuration.

The endoscope 100 can include a light source 230. In some examples, the light source 230 extends around the optical sensor 120. For instance, the light source 230 is located at a periphery of the optical sensor 120. In another example, the light source 230 can extend around (e.g., surrounds, rings, encompasses, or the like) the optical sensor 120. In yet another example, the light source 230 is adjacent to the optical sensor 120. The light source 230 can illuminate the distal field of view for observation with the optical sensor 120.

FIG. 3 shows another end view of the first end of the endoscope of FIG. 2. FIG. 3 shows the working channel 140 can have an expanded configuration, such as in which the channel wall 200 can be displaced relative to the first position (shown in FIG. 2). The working channel 140 can have a second cross-sectional area (shown with dashed lines in FIG. 3) in the expanded configuration. The second cross-sectional area can be larger than the first cross-sectional area. In another example, the channel wall 200 is in an expanded state in FIG. 3. For instance, the channel wall 200 can stretch (e.g., elastically deform, change shape, or the like) to accommodate an instrument in the working channel 200. Accordingly, in some examples, the working channel is expanded passively with the reception of the instrument within the working channel. In other examples, the working channel 140 is expanded actively, for instance using one or more inflatable balloons to expand the working channel 140.

The endoscope 100 with the expandable and collapsible working channel 140 can be structured or otherwise configured to ease access to the anatomy (e.g., a urethra, a bladder via the urethra, or the like) and/or minimize frictional engagement with anatomy (e.g., walls of a urethra, or the like) of a patient, such as during insertion. Accordingly, the endoscope 100 can minimize trauma to the anatomy. Thus, the endoscope 100 can help reduce or minimize patient discomfort, the risk of one or more of bleeding, developing scar tissue, or other trauma to tissue proximate the endoscope 100.

For example, the distal tip 130 can have a lateral tip dimension in a direction that is orthogonal to a longitudinal axis of the elongated endoscope device 100. The lateral tip dimension can be 3.8 millimeters with the working channel 140 in the collapsed configuration. The lateral tip dimension can be less than 5.5 millimeters with the working channel 140 in the expanded configuration. The working channel 140 can have a lateral inner dimension in a direction that is orthogonal to the longitudinal axis of the elongated endoscope device. For instance, the inner dimension of the working channel can be 2.2 millimeters in the collapsed configuration. In another example, the inner dimension of the working channel can be 0 millimeters in the collapsed configuration (e.g., the working channel can fully collapse, or the like). In another example, the inner dimension of the working channel can be within a range of 0.01 millimeters to 10 millimeters.

In an approach, an endoscope can have an outer diameter of 5.5 millimeters. In an example, the endoscope 100 having an expandable and collapsible working channel 140 can have an outer diameter of 3.8 millimeters with the working channel 140 in the expanded configuration. Thus, the expandable and collapsible working channel 140 can reduce overall size of the endoscope 100. Reducing overall size of the endoscope 100 can minimize trauma to anatomy.

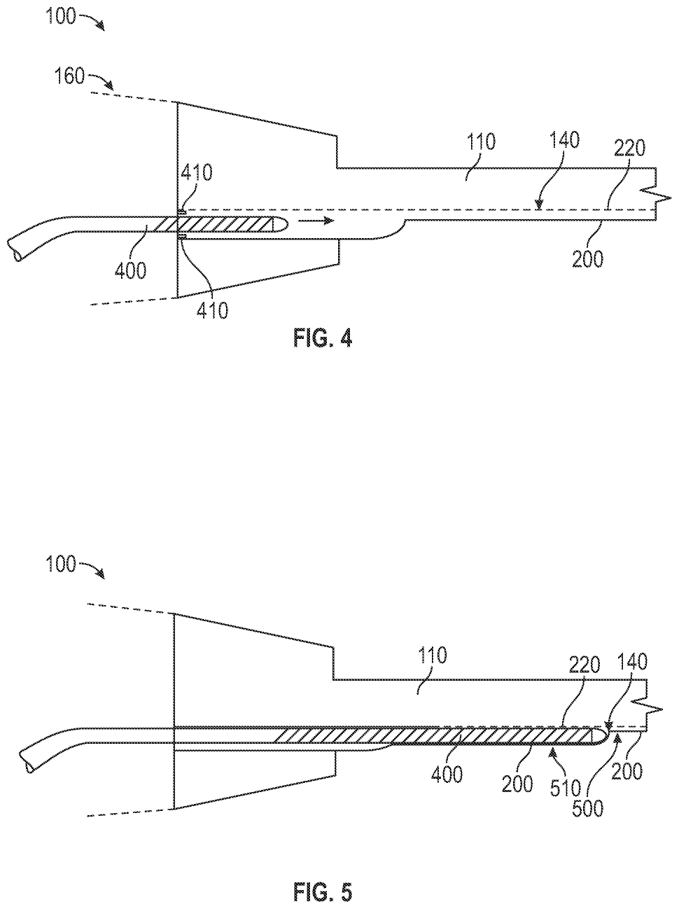

FIG. 4 shows a schematic diagram of the endoscope of FIG. 2. As mentioned, the working channel 140 can receive an instrument 400 (e.g., a scalpel, scissors, forceps, guidewire, laser fiber, or the like). For instance, the control section 160 can receive the instrument at the biopsy port 170 (shown in FIG. 1) and the biopsy port 170 can communicate with the working channel 140.

The working channel 140 can expand with reception of the instrument 400 within the working channel 140. For instance, the instrument 400 inserted into the working channel 140 can engage with one or more of the channel wall 200 or the socket wall 220 to expand the working channel 140. The engagement of the instrument with one or more of the channel wall 200 or the socket wall 220 displaces the channel wall relative to the instrument socket 220. For instance, the instrument can be compressed between the channel wall 200 and the socket wall 220. In another example, the working channel 140 expand conformally to the instrument 400 with the instrument received in the working channel 140. In another example, the instrument socket 210 can help guide the instrument 400 through the working channel 140. For instance, engagement of the instrument 400 with the socket wall 220 guides the instrument 400 through the working channel 140.

FIG. 5 shows another schematic diagram of the endoscope 100 of FIG. 2. For instance, FIG. 5 shows the instrument 400 can engage with one or more of the channel wall 200 or the socket wall 220. Engagement of the instrument 400 with the channel wall 200 or the socket wall 220 can expand the working channel 140. For example, FIG. 5 shows the channel wall 200 can have a first portion 500 in the first position (also shown in FIG. 4). A second portion 510 of the channel wall 200 can be displaced relative to the first position. For instance, engagement of the instrument 400 with one or more of the channel wall 200 or the socket wall 220 can displace the channel wall relative to the first position. In another example, FIG. 5 shows the channel wall 200 can be in an expanded (e.g., stretched, deformed, distended, enlarged, or the like) state. For instance, the channel wall 200 expands to accommodate the instrument 400 within the working channel.

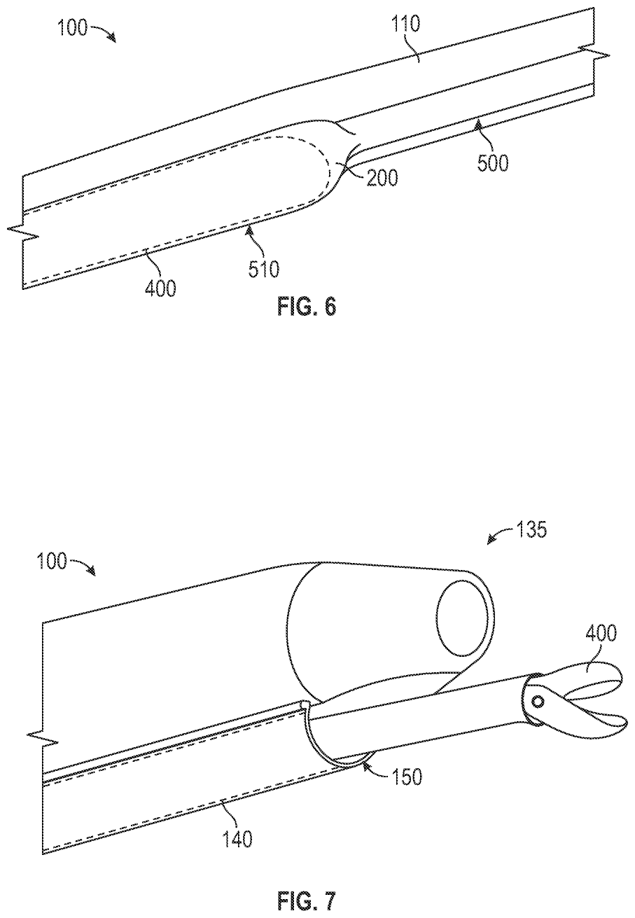

FIG. 6 shows a side view of the endoscope 100 of FIG. 2. In an example, the second portion 510 of the channel wall 200 can be displaced relative to the first portion 500 of the channel wall 200. For instance, FIG. 6 shows the instrument 400 can be engaged with the channel wall 200 (with the instrument received in the working channel 140, shown in FIG. 5). In another example, FIG. 6 shows the channel wall can be in an expanded (e.g., stretched, deformed, distended, enlarged, unfurled, or the like) state. FIG. 6 shows expansion of the channel wall 200 with the instrument 400 received in the working channel 140 (shown in FIG. 5).

FIG. 7 shows yet another end view of the first end 135 of the endoscope 100 of FIG. 2. FIG. 7 shows the instrument 400 can project from the channel opening 150 of the working channel 140. Additionally, FIG. 7 shows the working channel 140 expanded to accommodate the instrument 400.

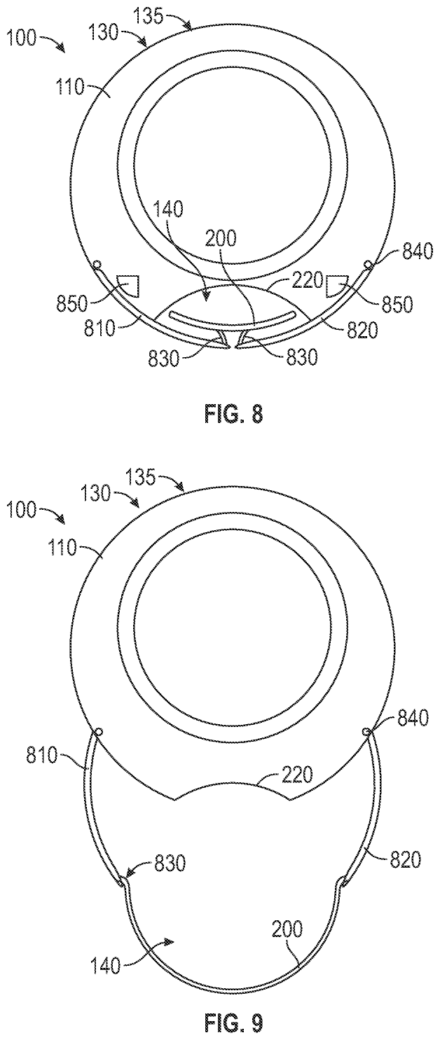

FIGS. 8 and 9 show end views of another example of the first end 135 of the endoscope 100. The endoscope 100 can include one or more working channel members 800. For instance, the endoscope can include one or more of a first door 810 or a second door 820. The endoscope 100 can include a flap 830. The endoscope 100 can include the channel wall 200. FIG. 8 shows the doors 810, 820 are closed with the working channel in the collapsed configuration. FIG. 9 shows the doors 810, 820 are open, and the channel wall 200 is displaced (e.g., displaced relative to the socket wall 220) relative to the position of channel wall 200 shown in FIG. 9.

One or more of the working channel members 800 (e.g., doors 810, 820; flap 830; channel wall 200; or the like) can be flexible, for example flexible in comparison to other portions of the endoscope 100. For instance, one or more of the working channel members 800 can comprise an elastic (or super-elastic) material (e.g., low durometer silicone rubber, elastic in comparison to the socket wall 220, or the like). In an example, the working channel members 800 can include a low durometer silicone rubber. In another example, the channel wall 220 (or other portions of the endoscope body 110) can be more rigid than one or more of working channel components 800. The endoscope body 110 can have a first durometer that can be greater than a second durometer of one or more of the working channel members 800. One or more of the working channel members 800 can couple with the endoscope body 110 using a living hinge.

One or more of the working channel members 800 can be unfurled, for instance with the working channel in the expanded configuration. For instance, FIG. 8 shows an example of the channel wall furled, such as when the working channel 140 is in a collapsed state. FIG. 9 shows an example of at least the channel wall 200 unfurled, such as when the working channel 140 is in an expanded state.

The working channel members 800 can be unfurled passively. For instance, the reception of the instrument 400 (shown in FIG. 5) within the working channel 140 can unfurl the working channel members 800. In another example, the instrument 400 engages with one or more of the working channel members 800 or the socket wall 220 to unfurl the working channel members 800. In yet another example, the working channel members 800 can be unfurled actively. For instance, the working channel members 800 can be unfurled using one or more balloons. The balloons can be inflated to unfurl the working channel members 800.

One or more of the doors can be hinged. For instance, one or more of the first door 810 or the second door 820 can be connected with the endoscope body at the hinge 840. The doors 810, 820 can rotate about the hinge 840 to allow the working channel members 800 to unfurl. The hinge 840 can be a living hinge.

The endoscope 100 can comprise an exterior hydrophilic coating to help reduce friction between the endoscope 100 and tissue of the patient. The working channel 140 can include a hydrophobic or hydrophilic coating. The hydrophobic or hydrophilic coating can help smooth engagement between an instrument and the working channel 140. For instance, the hydrophobic or hydrophilic coating can help reduce friction during translational movement of the instrument within the working channel 140.

In a further example, the endoscope 100 can comprise a drug eluting component. For instance, the endoscope 100 can elute a substance to numb tissue contacted by the endoscope 100. This can help enhance comfort of the patient undergoing cystoscopy.

Referring to FIG. 8, the endoscope 100 can include one or more fluid lines 850. The fluid lines 850 can supply one or more fluids (e.g., saline, water, air, gel, or the like) to the distal tip 130 of the endoscope 100. For example, the fluid can be discharged from the endoscope 100 at the distal tip 130.

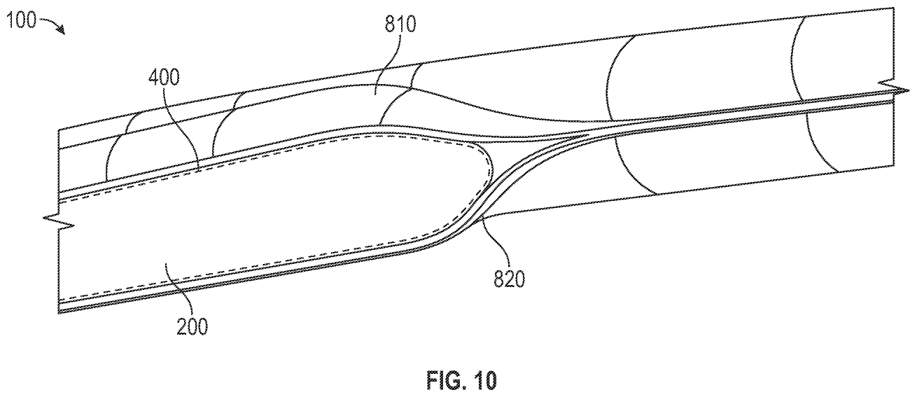

FIG. 10 shows a side view of the endoscope of FIG. 8. In an example, the instrument 400 is received in the working channel 140 (shown in FIGS. 8 and 9), and the working channel members 800 are unfurled. For instance, the doors 810, 820 can open to accommodate the instrument within the working channel 140.

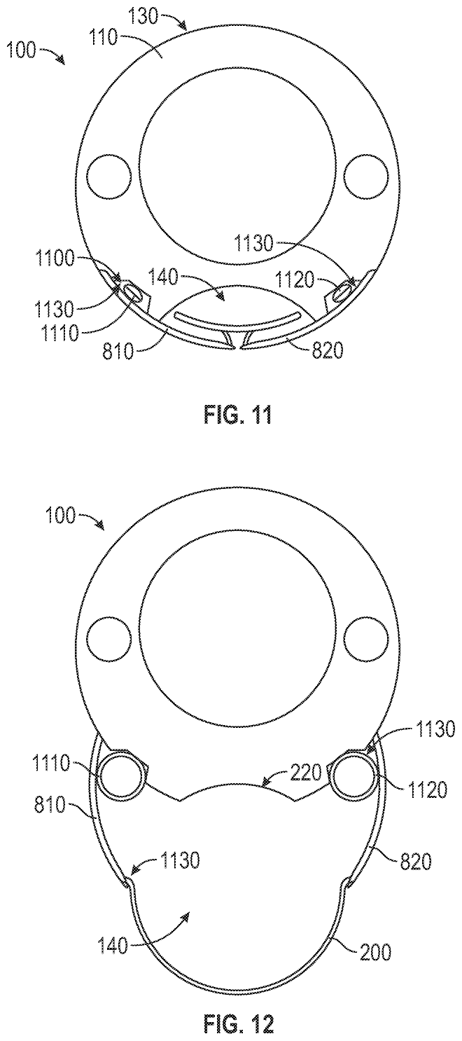

FIG. 11 shows an end view of yet another example of the endoscope 100. The endoscope 100 can include one or more balloons 1100. For example, FIG. 11 shows first and second balloons 1110, 1120 in a deflated configuration. One or more of the balloons 1110, 1120 can be received in a balloon cavity 1130 of the endoscope body 110. The balloon cavity 1130 can extend along a length of the endoscope body (e.g., along a longitudinal axis of the endoscope body 110, or the like).

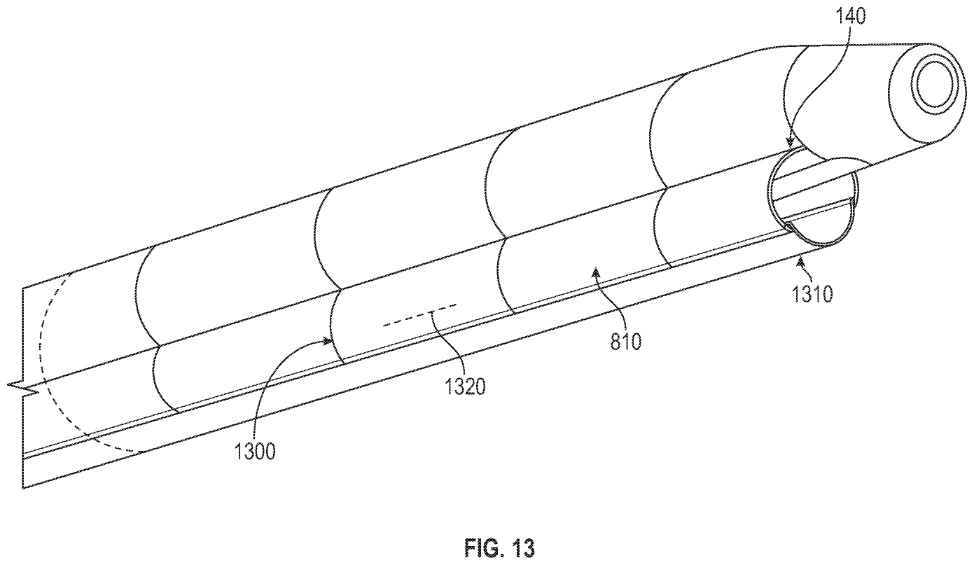

FIG. 12 shows the first and second balloons 1110, 1120 in an inflated configuration. The balloons 1100 can be fluid-inflated, such as to help expand the working channel 140. For instance, the one or more balloons 1100 can be located adjacent to respective doors (e.g., first and second doors 810, 820; or the like), such as to help open the respective doors and unfurl the channel wall 200. A fluid such as saline can be supplied to the balloons 1100 to inflate the balloons such as to expand the working channel 140. The saline can be pumped into the balloons 1100 from an external pump or other fluid source that can be fluidly coupled to a fluid port on a proximal portion of the endoscope 100, from which one or more fluid communication conduits are provided extending to the respective balloons 1100 to expand the balloons 1100. Expanding the balloons 1100 can help expand the working channel 140. In another example, wires (or rods) can be used to expand the balloons 1100.

The balloons 1100 can also help reduce or minimize force applied on an instrument inserted into the working channel 140. The balloons can be used to expand the working channel 140 without inserting an instrument into the working channel. This can place the working channel 140 in the expanded configuration (using the balloons) and ready to receive an instrument. Thus, the balloons 1100 can expand the working channel before inserting the instrument.

As mentioned, a fluid (e.g., gas, liquid, or the like) such as saline can be used to expand the balloons 1100 (and correspondingly expand the working channel 140). The saline can be discharged from the distal tip 130 of the endoscope. For instance, the saline can be used to irrigate or flush the anatomy of a patient. For example, the saline can be discharged from the balloons 1100 at the distal tip 130. In one example, the pressure is monitored in the balloons 1100 to facilitate inflation of the balloons 1100 while saline is discharged from the ends of the balloons 1100. The endoscope 100 can apply suction to remove the saline from the anatomy. The suction can be applied to the working channel 140 to remove the saline from the anatomy.

FIG. 13 shows another example of the first end 135 of the endoscope 100. The working channel 140 can normally be initially closed. For instance, FIG. 13 shows one or more clips 1300 that can hold the working channel 140 in the collapsed configuration. For instance, the clips 1300 can hold the doors 810, 820 closed. The clips 1300 can supply a biasing force to hold the working channel in the collapsed configuration. Inflating and expanding the balloons 1100 can overcome the biasing force of the clips 1300 to expand the working channel 140 (shown in FIG. 12).

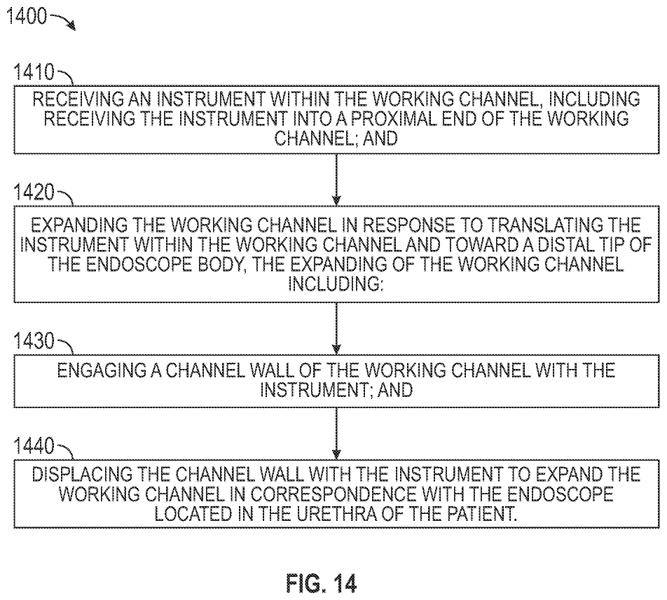

FIG. 14 shows an example of a method 1400 for using an endoscope, for instance the endoscope 100. The endoscope can be used within a urethra of a patient. The endoscope 100 can have an endoscope body 110 and an expandable and collapsible working channel 140. The method 1400 can include at 1410 receiving an instrument within the working channel, including receiving the instrument 400 into a proximal end 130 of the working channel. In another example, the method 1400 includes at 1420 expanding the working channel 140 in response to translating the instrument 400 within the working channel 400 and toward a distal tip 130 of the endoscope body 110. At 1430, the method 1400 can include expanding of the working channel by engaging a channel wall 200 of the working channel 140 with the instrument 400. At 1440, the method 1400 can include expanding the working channel by displacing the channel wall 200 with the instrument 400 to expand the working channel 140 in correspondence with the endoscope 100 located in the urethra of the patient.

In another example, the method 1400 can include observing the instrument 400 with an optical sensor 120 located at the distal tip 130 of the endoscope body 110. In yet another example, the method 1400 can include translating the instrument within the working channel and away from the distal tip 130 of the endoscope body 110, including disengaging the instrument 400 from the channel wall 200. In still yet another example, the method 1400 can include exposing the instrument 400 at the distal tip 130 of the endoscope body 110, including the instrument 400 projecting from a channel opening 150 of the working channel 140.

### VARIOUS NOTES AND EXAMPLES

Example 1 is an endoscope configured for insertion into a urethra of a patient, the endoscope comprising: an endoscope body extending between a proximal end and a distal end and having a distal tip; an optical sensor located at the distal tip and configured to provide a distal field of view from the distal tip; and an expandable and collapsible working channel configured for reception of an instrument, the working channel extending along the endoscope body to a channel opening located at the distal tip, wherein the working channel has a collapsed configuration and an expanded configuration, and wherein: in the collapsed configuration, the working channel is collapsed and a channel wall is in a first position relative to the endoscope body, and the working channel has a first cross-sectional area; and in the expanded configuration, the working channel is expanded and the channel wall is displaced relative to the first position and the working channel has a second cross-sectional area larger than the first cross-sectional area.

In Example 2, the subject matter of Example 1 optionally includes wherein the channel wall is elastically deformed in the expanded configuration.

In Example 3, the subject matter of Example 2 optionally includes wherein the channel wall is unfurled in the expanded configuration.

In Example 4, the subject matter of any one or more of Examples 1-3 optionally include wherein the channel wall is unfurled in the expanded configuration.

In Example 5, the subject matter of any one or more of Examples 1-4 optionally include wherein the channel wall is configured to conformally expand in response to insertion of the instrument received in the working channel.

In Example 6, the subject matter of any one or more of Examples 1-5 optionally include one or more working channel members including the channel wall, and the working channel features are configured to unfurl with the working channel in the expanded configuration.

In Example 7, the subject matter of Example 6 optionally includes wherein the one or more working channel members include at least one door, and the at least one door is configured to open with the working channel in the expanded configuration.

In Example 8, the subject matter of any one or more of Examples 1-7 optionally include wherein the endoscope body defines an instrument socket, and the working channel is between a socket wall of the instrument socket and the channel wall.

In Example 9, the subject matter of Example 8 optionally includes wherein the channel wall is relatively more proximate the socket wall in the collapsed configuration, and the channel wall is relatively more remote from the socket wall in the expanded configuration.

In Example 10, the subject matter of any one or more of Examples 1-9 optionally include the instrument received in the working channel, and the instrument projects from the distal tip at the channel opening.

In Example 11, the subject matter of any one or more of Examples 1-10 optionally include wherein the instrument includes one or more of a scalpel, scissors, forceps, guidewire, or laser fiber.

In Example 12, the subject matter of any one or more of Examples 1-11 optionally include a fluid supply line.

In Example 13, the subject matter of any one or more of Examples 1-12 optionally include one or more inflatable balloons configured to transition the working channel between the collapsed configuration and the expanded configuration.

Example 14 is a cystoscopy method of using an endoscope within a urethra of a patient, the endoscope having an endoscope body and an expandable and collapsible working channel, the method comprising: receiving an instrument within the working channel, including receiving the instrument into a proximal end of the working channel; and expanding the working channel in response to translating the instrument within the working channel and toward a distal tip of the endoscope body, the expanding of the working channel including: engaging a channel wall of the working channel with the instrument; and displacing the channel wall with the instrument to expand the working channel in correspondence with the endoscope located in the urethra of the patient.

In Example 15, the subject matter of Example 14 optionally includes observing the instrument with an optical sensor located at the distal tip of the endoscope body.

In Example 16, the subject matter of any one or more of Examples 14-15 optionally include translating the instrument within the working channel and away from the distal tip of the endoscope body, including disengaging the instrument from the channel wall.

In Example 17, the subject matter of any one or more of Examples 14-16 optionally include exposing the instrument at the distal tip of the endoscope body, the instrument projecting from a channel opening of the working channel.

Each of these non-limiting examples can stand on its own, or can be combined in various permutations or combinations with one or more of the other examples.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An endoscope configured for insertion into a urethra of a patient, the endoscope comprising:
an endoscope body extending between a proximal end and a distal end and having a distal tip;
an optical sensor located at the distal tip and configured to provide a distal field of view from the distal tip; and
an expandable and collapsible working channel configured for reception of an instrument, the working channel extending along the endoscope body to a channel opening located at the distal tip, wherein the working channel has a collapsed configuration and an expanded configuration, and wherein:
in the collapsed configuration, the working channel is collapsed and a channel wall is in a first position relative to the endoscope body, and the working channel has a first cross-sectional area; and
in the expanded configuration, the working channel is expanded and the channel wall is displaced relative to the first position and the working channel has a second cross-sectional area larger than the first cross-sectional area.

2. The endoscope of claim 1, wherein the channel wall is elastically deformed in the expanded configuration.

3. The endoscope of claim 2, wherein the channel wall is unfurled in the expanded configuration.

4. The endoscope of claim 1, wherein the channel wall is unfurled in the expanded configuration.

5. The endoscope of claim 1, wherein the channel wall is configured to conformally expand in response to insertion of the instrument received in the working channel.

6. The endoscope of claim 1, further comprising one or more working channel members including the channel wall, and the working channel features are configured to unfurl with the working channel in the expanded configuration.

7. The endoscope of claim 6, wherein the one or more working channel members include at least one door, and the at least one door is configured to open with the working channel in the expanded configuration.

8. The endoscope of claim 1, wherein the endoscope body defines an instrument socket, and the working channel is between a socket wall of the instrument socket and the channel wall.

9. The endoscope of claim 8, wherein the channel wall is relatively more proximate the socket wall in the collapsed configuration, and the channel wall is relatively more remote from the socket wall in the expanded configuration.

10. The endoscope of claim 1, further comprising the instrument received in the working channel, and the instrument projects from the distal tip at the channel opening.

11. The endoscope of claim 1, wherein the instrument includes one or more of a scalpel, scissors, forceps, guidewire, or laser fiber.

12. The endoscope of claim 1, further comprising a fluid supply line.

13. The endoscope of claim 1, further comprising one or more inflatable balloons configured to transition the working channel between the collapsed configuration and the expanded configuration.

14. A cystoscopy method of using an endoscope within a urethra of a patient, the endoscope having an endoscope body and an expandable and collapsible working channel, the method comprising:
receiving an instrument within the working channel, including receiving the instrument into a proximal end of the working channel; and
expanding the working channel in response to translating the instrument within the working channel and toward a distal tip of the endoscope body, the expanding of the working channel including:
engaging a channel wall of the working channel with the instrument; and
displacing the channel wall with the instrument to expand the working channel in correspondence with the endoscope located in the urethra of the patient.

15. The cystoscopy method of claim 14, further comprising observing the instrument with an optical sensor located at the distal tip of the endoscope body.

16. The cystoscopy method of claim 14, further comprising translating the instrument within the working channel and away from the distal tip of the endoscope body, including disengaging the instrument from the channel wall.

17. The cystoscopy method of claim 14, further comprising exposing the instrument at the distal tip of the endoscope body, the instrument projecting from a channel opening of the working channel.
